(19) United States
(12) Patent Application Publication
(10) Pub. No.: US 2001/0026919 A1
(43) Pub. Date: Oct. 4, 2001

(54) Nucleic acid assays employing universal arrays

(76) Inventors: Alex Chenchik, Palo Alto, CA (US); Grigoriy S. Tchaga, Newark, CA (US); Peter N. Simonenko, Mountain View, CA (US)

Correspondence Address: Bret E. Field, BOZICEVIC, FIELD & FRANCIS LLP, Suite 200, 200 Middlefield Road, Menlo Park, CA, 94025, US

(21) Appl. No.: 09/752,293

(22) Filed: Dec. 28, 2000

Related U.S. Application Data
(60) Provisional application No. 60/181,366, filed on Feb. 8, 2000.

(51) Int. Cl. C12Q 1/68

(52) U.S. Cl. 435/6

(57) ABSTRACT

Hybridization assays, as well as kits, primers and arrays for use in practicing the same, are provided. In the subject assays, a population of tagged target nucleic acids generated from a population of tagged gene specific primers is contacted with an array of tag complements under hybridization conditions and the presence of any resultant hybridized tag target nucleic acid-tag complement structures is detected. The subject arrays find use in a number of different applications, e.g. differential gene expression analysis.

### CROSS REFERENCE TO RELATED APPLICATIONS

Pursuant to 35 U.S.C. §119 (e), this application claims priority to the filing date of the U.S. Provisional Patent Application Ser. No. 60/181,366 filed Feb. 8, 2000, the disclosure of which is herein incorporated by reference.

### INTRODUCTION

1. Technical Field

The field of this invention is nucleic acid arrays.

2. Background of the Invention

Nucleic acid arrays have become an increasingly important tool in the biotechnology industry and related fields. Nucleic acid arrays, in which a plurality of nucleic acids are deposited onto a solid support surface in the form of an array or pattern, find use in a variety of applications, including drug screening, nucleic acid sequencing, mutation analysis, and the like.

One important use of nucleic acid arrays is in the analysis of differential gene expression, where the expression of genes in different cells, normally a cell of interest and a control, is compared and any discrepancies in expression are identified. In such assays, the presence of discrepancies indicates a difference in the classes of genes expressed in the cells being compared.

In methods of differential gene expression, arrays find use by serving as a substrate to which is bound nucleic acid “probe” fragments. One then obtains “targets” from at least two different cellular sources which are to be compared, e.g. analogous cells, tissues or organs of a healthy and diseased organism. The targets are then hybridized to the immobilized set of nucleic acid “probe” fragments. Differences between the resultant hybridization patterns are then detected and related to differences in gene expression in the two sources. Generally, in differential gene expression applications, a given array must be customized in terms of the probes displayed on its surface for a given application, severely restricting the different types of application sin which the array may find use.

Arrays of tag complements or molecular bar codes have been described in the literature for various applications. For example, Shoemaker et al., Nature Genet. (1996) 14:450-456 describes an array of 20-mer tag complements and its use in the phenotypic analysis of yeast deletion mutants, where each deletion mutant is labeled with an oligonucleotide tag. U.S. Pat. No. 5,763,175 to Sydney Brenner describes the use of an array of arbitrary tag complements and its use in high throughput sequencing applications in which tags are attached to nucleic acids to be sequenced and then hybridized to the array of tag complements. WO 00/58516 describes an array of arbitrary nucleic acids probes and its use in genotyping applications, in which a collection of locus specific tagged oligonucleotides is used in conjunction with the array of arbitrary tag complements in a single base extension reaction. While the above references describe various formats of arrays of tag complements and certain applications, none of these references suggest the use of such arrays in differential gene expression analysis applications or provide any guidance or suggestion as to how one would employ such an array in a differential gene expression analysis protocol.

Because of the continually growing importance of differential gene expression analysis and the high cost of customized arrays used in such protocols, there is a desire to find lower cost arrays suitable for use in such applications.

Relevant Literature

U.S. patents of interest include: U.S. Pat. Nos. 5,143,854; 5,445,934; 5,556,752; 5,700,637; 5,763,175; 5,807,522; 5,863,722; and 5,994,076. Also of interest are: WO 97/24455; WO 98/53103; WO 99/35289; and WO 00/58516. References of interest include: Shoemaker et al., Nature Genet. (1996) 14: 450-456; Southern, et al. Nature Genet. (1999) 21:5-9; Lipshutz, et al., Nature Genet. 1999, 21:20-24; Duggan, et al., Nature Genet. (1999) 21:10-14; and Brown, P. O., Nature Genet (1999) 21:33-37.

### SUMMARY OF THE INVENTION

Hybridization assays, as well as kits, primers and arrays for use in practicing the same, are provided. In the subject assays, a population of tagged target nucleic acids generated from a population of tagged gene specific primers is contacted with an array of tag complements under hybridization conditions and the presence of any resultant hybridized tagged target nucleic acid-tag complement structures is detected. The subject arrays find use in a number of different applications, e.g., differential gene expression analysis.

### DEFINITIONS

The term “nucleic acids” used herein means a polymer composed of nucleotides, e.g. naturally occurring deoxyribonucleotides or ribonucleotides, as well as synthetic mimetics thereof which are also capable of participating in sequence specific, Watson-Crick type hybridization reactions, such as is found in peptide nucleic acids, etc.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “target nucleic acid” means a nucleic acid that corresponds to a nucleic acid of interest present in a sample being assayed, i.e. a nucleic acid that is identical to or is the complement of a nucleic acid of interest, e.g. mRNA, a domain of genomic DNA, etc.

The term “tag” refers to a nucleic acid which has a sequence that is substantially non-homologous to, i.e., in many embodiments has less has less than about 50%, usually less than about 40% and more usually less than about 30% sequence identity to, the target nucleic acid to which it is attached in the subject methods.

The term “tag-complement” refers to a nucleic acid that hybridizes to a tag under stringent hybridization/working conditions.

The term “non-specific hybridization” refers to the non-specific binding or hybridization of a target nucleic acid to a nucleic acid present on the array surface, e.g. a long oligonucleotide probe of a probe spot on the array surface, a nucleic acid of a control spot on the array surface, and the like, where the target and the probe are not substantially complementary.

### DESCRIPTION OF THE SPECIFIC EMBODIMENTS

Hybridization assays, as well as kits, primers and arrays for use in practicing the same, are provided. In the subject assays, a population of tagged target nucleic acids generated using a population of tagged gene specific primers is contacted with an array of tag complements under hybridization conditions and the presence of any resultant hybridized tagged target nucleic acid-tag complement structures is detected. The subject arrays find use in a number of different applications, e.g. differential gene expression analysis. In further describing the subject invention, the subject methods are discussed first, followed by a review of representative applications in which the subject methods find use as well as a discussion of kits for use in practicing the subject methods.

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an” and “the” include plural reference unless the context clearly dictates otherwise. Unless defined otherwise, all technical and scientific terms used herein have the same meaning as commonly understood to one of ordinary skill in the art to which this invention belongs.

Methods

As summarized above, the subject invention provides methods for performing array based hybridization assays with a “universal array.” By “array based hybridization assay” is meant an assay or test protocol in which a nucleic acid array, i.e. a plurality of distinct probe nucleic acids stably associated or immobilized on the surface of a solid support (e.g. rigid or flexible solid support), is employed and one or more hybridization interactions occur, i.e. one or more specific Watson-Crick base pairing interactions between complementary nucleic acid molecules, i.e. probe nucleic acids immobilized on the array surface and target nucleic acids present in solution. For purposes of convenience in describing the invention, the assays are herein described in terms of hybridization interactions between probe and target nucleic acids, where the probe nucleic acids are those stably associated with the surface of the solid support and the target nucleic acids are the nucleic acids that hybridize to the array surface if their complement nucleic acid is present on the array surface as a probe nucleic acid. In other words, the subject invention provides methods of performing nucleic acid array hybridization assays between an array of probe nucleic acids stably associated with or immobilized on the surface of a solid support and a solution of target nucleic acids.

A feature of the subject invention is that, in practicing the subject array based hybridization assays, a population or plurality of distinct tagged target nucleic acids is contacted with an array of tag complements. As such, the target nucleic acids employed in the subject methods are tagged nucleic acids and the probe nucleic acids of the arrays employed in the subject methods are tag complements. In other words, in practicing the subject methods an array of a plurality of distinct tag complements is contacted with a population or plurality of tagged target nucleic acids. In addition, each tag and tag complement in a given population of tag-tag complement pairs employed in the subject assays is chosen to provide substantially uniform hybridization efficiency and substantially no cross-hybridization. In further describing this feature of the subject methods, the population of tagged target nucleic acids (and its preparation) will be described first, followed by a description of the tag complement arrays (and methods for their preparation). Finally, further detail regarding the hybridization efficiency and the low cross-hybridization characteristics of the tag-tag complements employed in the subject methods will be provided.

Population of Tagged Target Nucleic Acids and Methods for Its Production

As mentioned above, the subject methods employ a population of distinct tagged target nucleic acids. Of particular interest in many embodiments is the use of a population of distinct tagged targets of reduced complexity, where by reduced complexity is meant that the complexity of the tagged targets, i.e., the number of distinct targets of differing sequence in the population, is less than the complexity of the initial nucleic acid sample obtained from a biological source and from which the population of tagged targets is produced.

By population is meant a plurality, where the number of distinct target nucleic acids in a given population is generally at least about 10, usually at least about 20 and often at least about 50, wherein in many embodiments the number of distinct tagged target nucleic acids in a given population may be at least about 100, 200 or higher. In general, the number of distinct tagged target nucleic acids in a given population does not exceed about 10,000 and usually does not exceed about 2,000. For any given distinct tagged target nucleic acid in a population, its copy number may vary, but is generally at least about 1 in 10⁷ molecules, usually at least about 1 in 10⁶ molecules and more usually at least about 1 in 10⁵ molecules, where the copy number may be as high as 1 in 100 molecules or higher.

By tagged target nucleic acid is meant a nucleic acid that includes a target nucleic acid domain and a tag domain, where the two domains are covalently joined to each other, e.g. directly or through a linking group. In other words, the tagged target nucleic acid comprises a target nucleic acid domain covalently joined to a tag nucleic acid domain, either directly or through a linking group, where the linking group may or may not be cleavable, e.g. enzymatically cleavable (for example, it may include a restriction endonuclease recognized site), photo labile, etc.

Target Nucleic Acid Domain

The target nucleic acid domain is made up of a nucleic acid in which the sequence of nucleotides is a sequence (or the complement thereof) found in a nucleic acid of interest derived from a sample being assayed, e.g. an mRNA, a gene etc., which is present in a physiological sample. In other words, the target nucleic acid includes a stretch of nucleotide residues whose sequence is a sequence found in genomic DNA and/or in an mRNA present in the sample being assayed (or the complement thereof). For example, where one is interested in determining whether a particular gene is expressed in a cell sample of interest, the target nucleic acid domain of tagged target nucleic acids produced from the sample is one that has a stretch of nucleotide residues having a sequence that is found in or is the complement to a sequence in an mRNA present in the sample and/or the genomic DNA of the cell from which the sample was derived. As such, the target nucleic acid domain is one that corresponds to a gene of interest in the sample being assayed, where by “corresponds” is meant that it includes a sequence of nucleotides found in the gene of interest, i.e. either in the plus or minus strand. As such, a complement domain or sequence, i.e., complementary sequence, is present in the plus or minus strand to which the target sequence hybridizes under stringent conditions. The length of the target nucleic acid domain may vary greatly depending on the protocol employed to prepare it (where a representative protocol is provided below) and is typically less than the size of the initial mRNAs present in the nucleic acid sample from which it is derived in expression profiling applications. As such, in many embodiments, the length of the target nucleic acid domain is at least about 5 nt, usually at least about 50 nt and more usually at least about 100 nt, where the length typically does not exceed about 3000 nt and in many embodiments does not exceed about 500 nt.

Tag Domain

The tag domain or component of the tagged target nucleic acids is a nucleic acid that has a sequence of nucleotides which is not found in the gene to which the tagged target nucleic acid corresponds, as described above. In other words, the tag component has a nucleotide sequence at least not found in the corresponding gene and preferably any other gene from an analyzed physiological source, such that the tag component will not hybridize under stringent conditions to a nucleic acid domain of the corresponding gene, e.g. the plus or minus strand of the corresponding gene, or a domain found in the mRNA transcribed therefrom, and preferably any other gene/mRNA as well. As the tag domain does not hybridize to a sequence in the corresponding gene or any other gene, the sequence of any 30, usually any 25 and more usually any 20 consecutive nucleotides in the tag will have a homology of less than about 80%, usually less than about 60% and more usually less than about 50% with any stretch of nucleotides of like length in the corresponding gene and preferably any other known gene. As such, the tag component has a nucleotide sequence that is unrelated to any sequence found in the corresponding gene or, preferably, any other known gene. In many preferred embodiments, all of the tag domains employed in a given method are selected to be non-homologous to any other known eukaryotic (e.g., mouse, human, drosophila, yeast, etc.) gene and often prokaryotic gene as well.

Any two tag domains are considered to be distinct if they include a stretch or domain of nucleotides of at least about 20 nt, usually at least about 15 nt and more usually at least about 10 nt which are non-homologous, i.e. have a homology as determined by BLAST using default settings of less than about 80%, preferably less than about 60% and more preferably less than about 50%.

The length of the tag component is sufficiently long to provide for hybridization under stringent conditions with its corresponding tag complement. As such, the length of the tag component generally ranges from about 10 to 70 nt in length, but is generally from about 18 to 60 and in many embodiments is from about 20 to 40 nucleotides in length. Generally, the tag component ranges in length from about 20 to 50 nt. The tag may be made up of ribonucleotides and deoxyribonucleotides as well as synthetic nucleotide residues that are capable of participating in Watson-Crick type or other similar type of complementary base pair interactions.

Preparation of Population of Tagged Target Nucleic Acids

Generally, a population of tagged gene specific primers are employed to generate the population of tagged target nucleic acids. A number of different tagged gene specific primer based protocols may be employed, where representative gene specific primer based protocols are described in detail below.

In gene specific primer based protocols, a set (i.e. pool, mixture, collection) of a representational number of tagged gene specific primers is used to generate the population of tagged target nucleic acids, where the population of tagged target nucleic acids is typically labeled, from a sample of nucleic acids, usually ribonucleic acids (RNAs), more commonly mRNA.

As the subject sets comprise a representational number of primers, the total number of different primers in any given set will be only a fraction of the total number of different or distinct RNAs in the sample, where the total number of primers in the set will generally not exceed 80%, usually will not exceed 50% and more usually will not exceed 20% of the total number of distinct RNAs, usually the total number of distinct messenger RNAs (mRNAs), in the sample. Any two given RNAs in a sample will be considered distinct or different if they comprise a stretch of at least 100 nucleotides in length in which the sequence similarity is less then 98%, as determined using the FASTA program (default settings). As the sets of gene specific primers comprise only a representational number of primers, with physiological sources comprising from 5,000 to 50,000 distinct RNAs, the number of different gene specific primers in the set of gene specific primers will typically range from about 20 to 10,000, usually from 50 to 2,000 and more usually from 75 to 1500.

Each of the tagged gene specific primers of the sets described above contains a tag domain and a primer domain, where the two domains are covalently joined to one another, either directly or through a linking group, as described supra. The tag domain is as described above. The primer domain is a domain of sufficient length to specifically hybridize to a distinct nucleic acid member of the sample, e.g. RNA or cDNA, where the length of the gene specific primers will usually be at least 8 nt, more usually at least 20 nt and may be as long as 25 nt or longer, but will usually not exceed 50 nt. The gene specific primers will be sufficiently specific to hybridize to complementary template sequence during the generation of labeled nucleic acids under conditions sufficient for primer extension synthesis, which conditions are known by those of skill in the art. In many embodiments, the tagged gene specific primers are used for cDNA synthesis from mRNA as a template. The number of mismatches between the gene specific primer sequences and their complementary template sequences to which they hybridize during the generation of labeled nucleic acids in the subject methods will generally not exceed 20%, usually will not exceed 10% and more usually will not exceed 5%, as determined by FASTA (default settings).

Generally, the sets of tagged gene specific primers will comprise tagged primers that correspond to at least 20, usually at least 50 and more usually at least 75 distinct genes as represented by distinct mRNAs in the sample, where the term “distinct” when used to describe genes is as defined above, where any two genes are considered distinct if they comprise a stretch of at least 100 nt in their RNA coding regions in which the sequence similarity does not exceed 98%, as determined by FASTA (default settings). In addition, each different gene specific primer in a given set typically hybridizes to a different mRNA in a sample, such that two different tagged gene specific primers do not hybridize to the same mRNA in a sample. In many embodiments, each different or distinct tagged gene specific primer hybridizes under stringent conditions to a different or distinct mRNA in a sample. As such, where a collection of tagged gene specific primers containes 75 distinct tagged gene specific primers, the collection of primers hybridizes under stringent conditions to 75 distinct mRNAs in sample.

The tagged gene specific primers may be synthesized by conventional oligonucleotide chemistry methods, where the nucleotide units may be: (a) solely nucleotides comprising the heterocyclic nitrogenous bases found in naturally occurring DNA and RNA, e.g. adenine, cytosine, guanine, thymine and uracil; (b) solely nucleotide analogs which are capable of base pairing under hybridization conditions in the course of DNA synthesis such that they function as the above nucleotides found in naturally occurring DNA and RNA, where illustrative nucleotide analogs include inosine, xanthine, hypoxanthine, 1,2-diaminopurine and the like; or (c) from combinations of the nucleotides of (a) and nucleotide analogs of (b), where with primers comprising a combination of nucleotides and analogues thereof, the number of nucleotide analogues in the primers will typically be less than 25 and more typically less than 5. The gene specific primers may comprise reporter or hapten groups, usually 1 to 2, which serve to improve hybridization properties and simplify detection procedure.

Depending on the particular point at which the gene specific primers are employed in the generation of the labeled nucleic acids, e.g. during first strand cDNA synthesis or following one or more distinct amplification steps, each gene specific primer may correspond to a particular RNA by being complementary or similar, where similar usually means identical, to the sequence of the particular RNA. For example, where the gene specific primers are employed in the synthesis of first strand cDNA, the gene specific primers will be complementary to regions of the RNAs to which they correspond.

In a preferred embodiment, each gene specific primer can be complementary to a sequence of nucleotides which is unique in the population of nucleic acids, e.g. mRNAs, with which the primers are contacted, or one or more of the gene specific primers in the set may be complementary to several nucleic acids in a given population, e.g. multiple mRNAs, such that the gene specific primer generates labeled nucleic acid when one or more of set of related nucleic acid species, e.g. species having a conserved region to which the primer corresponds, are present in the sample. Examples of such related nucleic acid species include those comprising: repetitive sequences, such as Alu repeats, Al repeats and the like; homologous sequences in related members of a gene-family; polyadenylation signals; splicing signals; or arbitrary but conserved sequences.

The gene specific primers of the sets of primers according to the subject invention are typically chosen according to a number of different criteria. In some embodiments of the invention, primers of interest for inclusion in the set include primers corresponding to genes which are typically differentially expressed in different cell types, in disease states, in response to the influence of external agents, factors or infectious agents, and the like. In other embodiments, primers of interest are primers corresponding to genes which are expected to be, or already identified as being, differentially expressed in different cell, tissue or organism types. Preferably, at least 2 different gene functional classes will be represented in the sets of gene specific primers, where the number of different functional classes of genes represented in the primer sets will generally be at least 3, and will usually be at least 5. In other words, the sets of gene specific primers comprise nucleotide sequences complementary to RNA transcripts of at least 2 gene functional classes, usually at least 3 gene functional classes, and more usually at least 5 gene functional classes. Gene functional classes of interest include oncogenes; genes encoding tumor suppressors; genes encoding cell cycle regulators; stress response genes; genes encoding ion channel proteins; genes encoding transport proteins; genes encoding intracellular signal transduction modulator and effector factors; apoptosis related genes; DNA synthesis/recombination/repair genes; genes encoding transcription factors; genes encoding DNA-binding proteins; genes encoding receptors, including receptors for growth factors, chemokines, interleukins, interferons, hormones, neurotransmitters, cell surface antigens, cell adhesion molecules etc.; genes encoding cell-cell communication proteins, such as growth factors, cytokines, chemokines, interleukins, interferons, hormones etc.; and the like. Less preferred are gene specific primers that are subject to formation of strong secondary structures with less than −10 kcal/mol; comprise stretches of homopolymeric regions, usually more than 5 identical nucleotides; comprise more than 3 repetitive sequences; have high, e.g. more than 80%, or low, e.g. less than 30%, GC content etc.

The particular genes represented in the set of gene specific primers will necessarily depend on the nature of physiological source from which the RNAs to be analyzed are derived. For analysis of RNA profiles of eukaryotic physiological sources, the genes to which the gene specific primers correspond will usually be Class II genes which are transcribed into RNAs having 5′ caps, e.g. 7-methyl guanosine or 2,2,7-trimethylguanosine, where Class II genes of particular interest are those transcribed into cytoplasmic mRNA comprising a 7-methyl guanosine 5′ cap and a polyA tail.

For analysis of RNA profiles of mammalian physiological sources, as described below, of particular interest are gene specific primers corresponding to the functional gene classes listed above. In many embodiments of interest, the gene specific primers are primers For analysis of RNA profiles of human physiological sources, the gene specific primers are primers corresponding to those genes (and specific capable of producing target capable of hybridizing to those specific regions of the genes) as listed in the following patents and patent applications, the disclosures of which are herein incorporated by reference: U.S. Pat. No. 5,994,076; U.S. application Ser. No. 09/053,375; U.S. application Ser. No. 09/442,589; U.S. application Ser. No. 09/440,302; U.S. application Ser. No. 09/454,226; U.S. application Ser. No. 09/442,366; U.S. application Ser. No. 09/442,385; U.S. application Ser. No. 09/442,384; U.S. application Ser. No. 09/221,480; U.S. application Ser. No. 09/222,432; U.S. application Ser. No. 09/222,436; U.S. application Ser. No. 09/222,437; U.S. application Ser. No. 09/222,251; U.S. application Ser. No. 09/221,481; U.S. application Ser. No. 09/222,256; U.S. application Ser. No. 09/222,248; U.S. application Ser. No. 09/222,253; U.S. application Ser. No. 09/441,920; and U.S. application Ser. No. 09/440,305.

Depending on the particular nature of the tagged target nucleic acid generation step of the subject methods, the tagged gene specific primers may be modified in a variety of ways. One way the gene specific primers may be modified is to include an anchor sequence of nucleotides, where the anchor is usually located 5′ of the gene specific portion of the primer before or after the tag portion and ranges in length from 10 to 50 nt in length, usually 15 to 40 nt in length. The anchor sequence may comprise a sequence of bases which serves a variety of functions, such as a sequence of bases which correspond to the sequence found in promoters for bacteriophage RNA polymerase, e.g. T7 polymerase, T3 polymerase, SP6 polymerase, and the like; arbitrary sequences which can serve as subsequent primer binding sites; for generating secondary structure or complimentary interaction with other sequences; and the like.

Turning now to the methods employing the above sets of tagged gene specific primers, the first step in the subject methods is to obtain a sample of nucleic acids, usually RNAs or nucleic acid derivatives thereof, like cDNA, amplified DNA, cRNA, etc., from a physiological source, usually a plurality of physiological sources, where the term plurality is used to refer to 2 or more distinct physiological sources. The physiological source of nucleic acids, e.g. RNAs, will typically be eukaryotic or prokaryotic, with physiological sources of interest including sources derived from single celled organisms such as bacteria and yeast and multicellular organisms, including plants and animals, particularly mammals, where the physiological sources from multicellular organisms may be derived from particular organs or tissues of the multicellular organism, or from isolated cells or subcellular/extracellular fractions derived therefrom. For prokaryotic sources (e.g., bacteria), the physiological sources may be different related strains of microorganisms (like pathogenic and non-pathogenic strains), organisms treated by different conditions (nutrition, toxic response, etc.); and the like. Thus, the physiological sources may be different cells from different organisms of the same species, e.g. cells derived from different humans, or cells derived from the same human (or identical twins) such that the cells share a common genome, where such cells will usually be from different tissue types, including normal and diseased tissue types, e.g. neoplastic, cell types. In obtaining the sample of RNAs to be analyzed from the physiological source from which it is derived, the physiological source may be subjected to a number of different processing steps, where such processing steps might include tissue homogenization, nucleic acid extraction and the like, where such processing steps are known to the those of skill in the art. Methods of isolating RNA from cells, tissues, organs or whole organisms are known to those of skill in the art and are described in Maniatis et al., Molecular Cloning: A Laboratory Manual (Cold Spring Harbor Press)(1989).

The next step in the subject methods is the generation of the population of tagged target nucleic acids from the initial sample, where the population is generally labeled and is representative of the nucleic acid, usually RNA, profile of the physiological source. As mentioned above, a set or pool of tagged gene specific primers is used to generate the labeled nucleic acids from the sample of RNAs. Since the subject sets or pools of primers are employed, a sub-population of nucleic acids is generated from the initial source, where the sub-population corresponds to only a portion or fraction of the initial nucleic acid source. As used herein, the term “target” refers to single stranded RNA, single stranded DNA and double stranded DNA, where the target is generally greater than 50 nt in length.

The set of tagged gene specific primers may be used either in first strand cDNA synthesis or following one or more synthesis/amplification steps. Furthermore, the actual synthesis of the labeled nucleic acids may be at the same step during which the sets of gene specific primers are employed, or the synthesis of the labeled nucleic acids may be one more steps subsequent to the step in which the sets of gene specific primers are employed. A feature of many preferred embodiments, however, is that the tagged gene specific primers are not employed in an amplification step, but solely in a primer extension step, which primer extension step does not include amplification. As such, while the overall protocol of tagged target nucleic acid generation may include one or more amplification steps, e.g. PCR steps, the tagged gene specific primers are not employed in any amplification step, but just in primer extension. As such, where the overall protocol includes amplification, non-tagged gene specific primers are employed in the amplification portion of the protocol.

In a first representative embodiment of the invention, the set of tagged gene specific primers is used to generate labeled first strand cDNA, where the labeled first strand cDNA is representative of the RNA profile of the physiological source being assayed. The labeled first strand cDNA is prepared by contacting the RNA sample with the primer set and requisite reagents under conditions sufficient for hybrid duplexes (i.e. double stranded primer complexes) to be produced followed by reverse transcription of the RNA template in the sample. Requisite reagents contacted with the primers and RNAs are known to those of skill in the art and will generally include at least an enzyme having reverse transcriptase activity and dNTPs in an appropriate buffer medium.

A variety of enzymes, usually DNA polymerases, possessing reverse transcriptase activity can be used for the first strand cDNA synthesis step. Examples of suitable DNA polymerases include the DNA polymerases derived from organisms selected from the group consisting of a thermophilic bacteria and archaebacteria, retroviruses, yeasts, Neurosporas, Drosophilas, primates and rodents. Preferably, the DNA polymerase will be selected from the group consisting of Moloney murine leukemia virus (M-MLV) as described in U.S. Pat. No. 4,943,531 and M-MLV reverse transciptase lacking RNaseH activity as described in U.S. Pat. No. 5,405,776 (the disclosures of which patents are herein incorporated by reference), human T-cell leukemia virus type I (HTLV-I), bovine leukemia virus (BLV), Rous sarcoma virus (RSV), human immunodeficiency virus (HIV) and Thermus aquaticus (Taq) or Thermus thermophilus (Tth) as described in U.S. Pat. No. 5,322,770, the disclosure of which is herein incorporated by reference. Suitable DNA polymerases possessing reverse transcriptase activity may be isolated from an organism, obtained commercially or obtained from cells which express high levels of cloned genes encoding the polymerases by methods known to those of skill in the art, where the particular manner of obtaining the polymerase will be chosen based primarily on factors such as convenience, cost, availability and the like.

The various dNTPs and buffer medium necessary for first strand cDNA synthesis through reverse transcription of the primed RNAs may be purchased commercially from various sources, where such sources include Clontech, Sigma, Life Technologies, Amersham, Roche, etc. Buffer mediums suitable for first strand synthesis will usually comprise buffering agents, usually in a concentration ranging from 10 to 100 mM which typically support a pH in the range 6 to 9, such as Tris-HCl, HEPES-KOH, etc.; salts containing monovalent ions, such as KCl, NaCl, etc., at concentrations ranging from 0-200 mM; salts containing divalent cations like MgCl₂, Mg(OAc)₂, MnCl₂, etc, at concentrations usually ranging from 1 to 10 mM; and additional reagents such as reducing agents, e.g. DDT, detergents, albumin and the like. The conditions of the reagent mixture will be selected to promote efficient first strand synthesis. Typically the set of primers will first be combined with the RNA sample at an elevated temperature, usually ranging from 50 to 95° C., followed by a reduction in temperature to a range between about 0 to 60° C., to ensure specific annealing of the primers to their corresponding RNAs in the sample. Following this annealing step, the primed RNAs are then combined with dNTPs and reverse transcriptase under conditions sufficient to promote reverse transcription and first strand cDNA synthesis of the primed RNAs, usually by incubating the reaction mixture at 37 to 60° C. for 0.5 to 1.0 hr. By using appropriate types of reagents, all of the reagents can be combined at once if the activity of the polymerase can be postponed or timed to start after annealing of the primer to the RNA.

In this embodiment, one of either the gene specific primers or dNTPs, preferably the dNTPs, will be labeled such that the synthesized cDNAs are labeled. By labeled is meant that the entities comprise a member of a signal producing system and are thus detectable, either directly or through combined action with one or more additional members of a signal producing system. Examples of directly detectable labels include isotopic and fluorescent moieties incorporated into, usually covalently bonded to, a nucleotide monomeric unit, e.g. dNTP or monomeric unit of the primer. Isotopic moieties or labels of interest include ³²P, ³³P, ³⁵S, ¹²⁵I, ³H, and the like. Fluorescent moieties or labels of interest include coumarin and its derivatives, e.g. 7-amino-4-methylcoumarin, aminocoumarin, bodipy dyes, such as Bodipy FL, cascade blue, fluorescein and its derivatives, e.g. fluorescein isothiocyanate, Oregon green, rhodamine dyes, e.g. texas red, tetramethylrhodamine, eosins and erythrosins, cyanine dyes, e.g. Cy3 and Cy5, macrocyclic chelates of lanthanide ions, e.g. quantum dye™, fluorescent energy transfer dyes, such as thiazole orange-ethidium heterodimer, TOTAB, etc. Labels may also be members of a signal producing system that act in concert with one or more additional members of the same system to provide a detectable signal. Illustrative of such labels are members of a specific binding pair, such as ligands, e.g. biotin, fluorescein, digoxigenin, antigen, polyvalent cations, chelator groups and the like, where the members specifically bind to additional members of the signal producing system, where the additional members provide a detectable signal either directly or indirectly, e.g. antibody conjugated to a fluorescent moiety or an enzymatic moiety capable of converting a substrate to a chromogenic product, e.g. alkaline phosphatase conjugate antibody; and the like. For each sample of RNA, one can generate labeled oligos with the same labels. Alternatively, one can use different labels for each physiological source, which provides for additional assay configuration possibilities, as described in greater detail below.

In a variation of the above embodiment, where desired one can generate labeled RNA instead of labeled first strand cDNA. In this embodiment, first strand cDNA synthesis is carried out in the presence of unlabeled dNTPs and unlabeled gene specific primers. However, the primers are optionally modified to comprise a promotor for an RNA polymerase, such as T7 RNA polymerase, T3 RNA polymerase, SP6 RNA polymerase, and the like. In this embodiment, following first strand cDNA synthesis, the resultant single stranded cDNA is then converted to double stranded cDNA, where the resultant double stranded cDNA comprises the anchor sequence comprising the promoter region. Conversion of the mRNA:cDNA hybrid following first strand synthesis can be carried out as described in Okayama & Berg, Mol. Cell. Biol. (1982) 2:161-170, and Gubler & Hoffman, Gene (1983) 25: 253-269, where briefly the RNA is digested with a ribonuclease, such as E. coli RNase H, followed by repair synthesis using a DNA polymerase like DNA polymerase I, etc., and E. coli DNA ligase. One may also employ the modifications of this basic method described in Wu, R, ed., Methods in Enzymology (1987), vol. 153 (Academic Press). Next, the double stranded cDNA is contacted with RNA polymerase and dNTPs, including labeled dNTPs, to produce linearly amplified labeled ribonucleic acids. For cDNA lacking the anchor sequence comprising a promoter region, a polymerase that does not need a promoter region but instead can initiate RNA strand synthesis randomly from cDNA, such as core fragment of E. Coli RNA polymerase, may be employed.

In another embodiment of the subject invention, the labeled nucleic acid generation step comprises one or more enzymatic amplification steps in which multiple DNA copies of the initial RNAs present in the sample are produced, from which multiple copies of the initial RNA or multiple copies of antisense or complementary RNA (aRNA or cRNA) may be produced, using the polymerase chain reaction, as described in U.S. Pat. No. 4,683,195, the disclosure of which is herein incorporated by reference, in which repeated cycles of double stranded DNA denaturation, oligonucleotide primer annealing and DNA polymerase primer extension are performed, where the PCR conditions may be modified as described in U.S. Pat No. 5,436,149, the disclosure of which is herein incorporated by reference.

In one embodiment involving enzymatic amplification, the set of gene-specific primers are employed in the generation of the first strand cDNA, followed by amplification of the first strand cDNA to produce amplified numbers of labeled cDNA. In this embodiment, as a set of gene-specific primers is employed in the first strand synthesis step, only a representative proportion of the total RNA in the sample is amplified during the subsequent amplification steps.

Amplification of the first strand cDNA can be conveniently achieved by using a CAPswitch™ oligonucleotide as described in U.S. Pat. No. 5,962,271, the disclosure of which is herein incorporated by reference. Briefly, the CAPswitch technology uses a unique CAPswitch™ oligonucleotide in the first strand cDNA synthesis followed by PCR amplification in the second step to generate a high yield of ds cDNA. When included in the first-strand cDNA synthesis reaction mixture, the CAPswitch™ oligonucleotide serves as a short extended template. When reverse transcriptase stops at the 5′ end of the mRNA template in the course of first strand cDNA synthesis it switches templates and continues DNA synthesis to the end of the CAPswitch™ oligonucleotide. The resulting ss cDNA incorporates at the 3′ end, sequence which is complimentary to complete 5′ end of the mRNA and the CAPswitch oligonucleotide sequence.

Of particular interest as the CAPswitch oligonucleotide are oligonucleotides having the following formula:

5′-dNₘ-rNₙ-3′

wherein:

dN represents a deoxyribonucleotide selected from among dAMP, dCMP, dGMP and dTMP;

m represents an integer 0 and above, preferably from 10 to 50;

rN represents a ribonucleotide selected from the group consisting of AMP, CMP, GMP and UMP, preferably GMP; and

n represents an integer 0 and above, preferably from 3 to 7.

The structure of the CAPswitch oligonucleotide may be modified in a number of ways, such as by replacement of 1 to 10 nucleotides with nucleotide analogs, incorporation of terminator nucleotides, such as 3′-amino NMP, 3′-phosphate NMP and the like, or non-natural nucleotides conjugating with CAP-binding polypeptides which can improve efficiency of the template switching reaction but still retain the main function of the CAPswitch oligonucleotide i.e. CAP-depended extension of full-length cDNA by reverse transcriptase using CAPswitch oligonucleotide as a template.

In using the CAPswitch oligonucleotide, first strand cDNA synthesis is carried out in the presence of a set of gene specific primers and a CAPswitch oligonucleotide, where the gene specific primers have been modified to comprise an arbitrary anchor sequence at their 5′ ends. The first strand cDNA is then combined with primer sequences complementary to: (a) all or a portion of the CAPswitch oligonucleotide and (b) the arbitrary anchor sequence of the gene specific primers and additional PCR reagents, such as dNTPs, DNA polymerase, and the like, under conditions sufficient to amplify the first strand cDNA. Conveniently, PCR is carried out in the presence of labeled dNTPs such that the resultant, amplified cDNA is labeled and serves as the labeled or target nucleic acid. Labeled nucleic acid can also be produced by carrying out PCR in the presence of labeled primers, where either or both the CAPswitch oligonucleotide complementary primer and anchor sequence complementary primer may be labeled. In yet an alternative embodiment, instead of producing labeled amplified cDNA, one may generate labeled RNA from the amplified ds cDNA, e.g. by using an RNA polymerase such as E. coli RNA polymerase, or other RNA polymerases requiring promoter sequences, where such sequences may be incorporated into the arbitrary anchor sequence.

Instead of using the set of gene specific primers in the first strand cDNA synthesis step followed by subsequent amplification of only a representative fraction of the total number of distinct RNA species in the sample, one may also amplify all of the RNAs in the sample and use the set of gene specific primers to generate labeled nucleic acid following amplification. This embodiment may find use in situations where the RNA of interest to be amplified is known or postulated to be in small amounts in the sample.

In this embodiment, first strand synthesis is carried out using: (a) an oligo dT or random primer that usually comprises an arbitrary anchor sequence at its 5′ end and (b) a CAPswitch oligonucleotide. During first strand synthesis the oligo(dT) anneals to the polyA tail of the mRNA in the sample and synthesis extends beyond the 3′ end of the RNA to include the CAPswitch oligonucleotide, yielding a first strand cDNA comprising an arbitrary sequence at its 5′ end and a region complementary to the CAPswitch oligonucleotide at its 3′ end. The length of the dT primer will typically range from 15 to 30 nts, while the arbitrary anchor sequence or portion of the primer will typically range from 15 to 25 nt in length.

Following first strand synthesis, the cDNA is amplified by combining the first strand cDNA with primers that correspond at least partially to the anchor sequence and the CAPswitch oligonucleotide primer under conditions sufficient to produce an amplified amount of the cDNA. Labeled nucleic acid is then produced by contacting the resultant amplified cDNA with a set of gene specific primers, a polymerase and dNTPs, where at least one of the gene specific primers and/or dNTPs are labeled.

The above representative protocols produce a population of tagged target nucleic acids, and generally labeled tagged target nucleic acids, from an initial nucleic acid source using a set of tagged gene specific primers. As mentioned above, while the overall protocol may include an amplification step, the tagged gene specific primers themselves are generally not employed in amplification, their use being limited to primer extension in many preferred embodiments of the subject invention.

Tag Complement Arrays

As summarized above, another feature of the subject methods is that an array of tag complements is employed. The tag complement arrays of the subject invention have a plurality of probe spots stably associated with or immobilized on a surface of a solid support. A feature of the subject tag complement arrays is that at least a portion of the probe spots, and preferably substantially all of the probe spots, on the array are tag complement probe spots, where each tag complement probe spot is generally made up of a number or plurality of identical nucleic acid probe molecules that include a tag complement domain.

Probe Spots of the Arrays

As mentioned above, a feature of the subject invention is the nature of the probe spots, i.e. that at least a portion of, and usually substantially all of, the probe spots on the array are made up of probe nucleic acid compositions of tag complements, i.e. generally at least a substantial portion of the probe spots are tag complement probe spots. Each tag complement probe spot on the surface of the substrate is made up of tag complement nucleic acid probes, where the spot may be homogeneous with respect to the nature of the probe molecules present therein or heterogenous, e.g. as described in U.S. patent application Ser. No. 60/104,179, the disclosure of which is herein incorporated by reference.

A feature of the subject tag complement probe compositions is that they are made up of probe molecules that include a tag complement domain and a substrate surface binding domain. By tag complement domain is meant a stretch or region of nucleotides that has a sequence which is the complement (i.e., has the complementary sequence) of a tag domain with which the subject array is used. In other words, the tag complement domain is a domain that hybridizes to a tag domain of a tagged target nucleic acid during in the subject methods. The length of the tag complement domain may vary, but is, in many embodiments, substantially the same length as the tag domain to which it hybridizes during practice of the subject methods, where by substantially the same length is meant that the magnitude of any difference in lengths typically does not exceed about 15 nt and usually does not exceed about 10 nt. As such, the length of the subject tag complement domains generally ranges from about 10 to 70 nt, usually from about 18 to 60 nt and more usually from about 20 to 40 nt. The sequence of nucleotides in the tag complement is chosen or selected based on a number of different parameters with respect to its corresponding tag, where these considerations and parameters are described in greater detail infra.

While in the broadest sense the probe molecules that make up the probe spots of the arrays employed in the subject methods may be any length, a feature of the probe compositions in the arrays employed in many of the embodiments of the subject invention is that the probe compositions are made up of long oligonucleotides. As such, the tag complement probes of the probe compositions range in length from about 50 to 150, typically from about 50 to 120 nt and more usually from about 60 to 100 nt, where in many preferred embodiments the probes range in length from about 65 to 85 nt. Such long oligonucleotides are further described in U.S. patent application Ser. No. 09/440,829, the disclosure of which is herein incorporated by reference.

In addition, the probe molecules of a given spot are chosen so that each tag complement probe molecule on the array is not homologous with any other distinct unique tag complement probe molecule present on the array, i.e. any other tag complement probe molecule on the array with a different base sequence. In other words, each distinct tag complement probe molecule of a probe composition corresponding to a first tag does not cross-hybridize (under stringent conditions) with, or have the same sequence as, any other distinct unique tag complement probe molecule of any probe composition corresponding to a different target, i.e. an oligonucleotide of any other oligonucleotide probe composition that is represented on the array. As such, the sense or anti-sense nucleotide sequence of each unique tag complement probe molecule of a probe composition will have less than 90% homology, usually less than 70% homology, and more usually less than 50% homology with any other different tag complement probe molecule of a probe composition on the array corresponding to a different tag, where homology is determined by sequence analysis comparison using the FASTA program using default settings.

The tag complement probe molecules of each probe composition, or at least the tag complement portion of these molecules, are further characterized as follows. First, they have a GC content of from about 35% to 80%, usually between about 40 to 70%. Second, they have a substantial absence of: (a) secondary structures, e.g. regions of self-complementarity (e.g. hairpins), structures formed by intramolecular hybridization events; (b) long homopolymeric stretches, e.g. polyA stretches, such that in any given homopolymeric stretch, the number of contiguous identical nucleotide bases does not exceed 4; (c) long stretches (more than 8 nt) characterized by or enriched by the presence of repeating motifs, e.g GAGAGAGA, GAAGAGAA, etc.; (d) long stretches (more than 8 nt) of homopurine or homopyrimidine rich motifs; and the like.

The tag complement probes of the subject invention may be made up solely of the tag complement sequence as described above, e.g. sequence designed or present which is intended for hybridization to the probe's corresponding tag, or may be modified to include one or more non-tag complementary domains or regions, e.g. at one or both termini of the probe, where these domains may be present to serve a number of functions, including attachment to the substrate surface, to introduce a desired conformational structure into the probe sequence, etc.

One optional domain or region that may be present at one or more both termini of the long oligonucleotide probes of the subject arrays is a region enriched for the presence of thymidine bases, e.g. an oligo dT region, where the number of nucleotides in this region is typically at least 3, usually at least 5 and more usually at least 10, where the number of nucleotides in this region may be higher, but generally does not exceed about 25 and usually does not exceed about 20, where at least a substantial portion of, if not all of, the nucleotides in this region include a thymidine base, where by substantial portion is meant at least about 50, usually at least about 70 and more usually at least about 90 number % of all nucleotides in the oligo dT region. Certain probes of this embodiment of the subject invention, i.e. those in which the T enriched domain is an oligo dT domain, may be described by the following formula:

Tₙ-Nₘ-Tₖ;

wherein:

T is dTMP;

Nₘ is the target specific sequence of the probe in which N is either dTMP, dGMP, dCMP or dAMP and m is from 15 to 50; and

n and k are independently from 0 to 15, where when present n and/or k are preferably 5 to 10.

In yet other embodiments and often in addition to the above described T enriched domains, the subject probes may also include domains that impart a desired constrained structure to the probe, e.g. impart to the probe a structure which is fixed or has a restricted conformation. In many embodiments, the probes include domains which flank either end of the target specific domain and are capable of imparting a hairpin loop structure to the probe, whereby the target specific sequence is held in confined or limited conformation which enhances its binding properties with respect to its corresponding target during use. In these embodiments, the probe may be described by the following formula:

Tₙ-Nₚ-Nₘ-Nₒ-Tₖ

wherein:

T is dTMP;

N is dTMP, dGMP, dCMP or dAMP;

m is an integer from 15 to 50;

n and k are independently from 0 to 15, where when present n and/or k are preferably 5 to 10, where in many embodiments k=n=5 to 10, more preferably 10; and

p and o are independently 5 to 20, usually 5 to 15, and more usually about 10, wherein in many embodiments p=o=5 to 15 and preferably 10;

such that Nₘ is the target specific sequence; and

Nₒ and Nₚ are self complementary sequences, e.g. they are complementary to each other, such that under hybridizing conditions the probe forms a hairpin loop structure in which the stem is made up of the Nₒ and Nₚ sequences and the loop is made up of the target specific sequence, i.e. Nₘ.

The tag complement probe compositions that make up each tag complement probe spot on the array will be substantially, usually completely, free of non-nucleic acids, i.e. the probe compositions will not include or be made up of non-nucleic acid biomolecules found in cells, such as proteins, lipids, and polysaccharides. In other words, the oligonucleotide spots of the arrays are substantially, if not entirely, free of non-nucleic acid cellular constituents.

The tag complement probes may be nucleic acid, e.g. RNA, DNA, or nucleic acid mimetics, e.g. nucleic acids that differ from naturally occurring nucleic acids in some manner, e.g. through modified backbones, sugar residues, bases, etc., such as nucleic acids comprising non-naturally occurring heterocyclic nitrogenous bases, peptide-nucleic acids, locked nucleic acids (see Singh & Wengel, Chem. Commun. (1998) 1247-1248); and the like. In many embodiments, however, the nucleic acids are not modified with a functionality which is necessary for attachment to the substrate surface of the array, e.g. an amino functionality, biotin, etc.

The tag complement probe spots made up of the tag complement probes as described above and present on the array may be any convenient shape, but will typically be circular, elliptoid, oval or some other analogously curved shape. The total amount or mass of tag complement probe molecules present in each spot will be sufficient to provide for adequate hybridization and detection of tagged target nucleic acid during the assay in which the array is employed. Generally, the total mass of nucleic acids in each spot will be at least about 0.1 ng, usually at least about 0.5 ng and more usually at least about 1 ng, where the total mass may be as high as 100 ng or higher, but will usually not exceed about 20 ng and more usually will not exceed about 10 ng. The copy number of all of the oligonucleotides in a spot will be sufficient to provide enough hybridization sites for tagged target molecule to yield a detectable signal, and will generally range from about 0.001 fmol to 10 fmol, usually from about 0.005 fmol to 5 fmol and more usually from about 0.01 fmol to 1 fmol. Where the spot is made up of two or more distinct tag complement probe molecules of differing sequence, the molar ratio or copy number ratio of different oligonucleotides within each spot may be about equal or may be different, wherein when the ratio of unique nucleic acids within each spot differs, the magnitude of the difference will usually be at least 2 to 5 fold but will generally not exceed about 10 fold.

Where the spot has an overall circular dimension, the diameter of the spot will generally range from about 10 to 5,000 μm, usually from about 20 to 1,000 μm and more usually from about 50 to 500 μm. The surface area of each spot is at least about 100 μm², usually at least about 200 μm² and more usually at least about 400 μm², and may be as great as 25 mm² or greater, but will generally not exceed about 5 mm², and usually will not exceed about 1 mm².

Additional Array Features

The arrays of the subject invention are characterized by having a plurality of probe spots as described above stably associated with the surface of a solid support. The density of probe spots on the array, as well as the overall density of probe and non-probe nucleic acid spots (where the latter are described in greater detail infra) may vary greatly. As used herein, the term nucleic acid spot refers to any spot on the array surface that is made up of nucleic acids, and as such includes both probe nucleic acid spots and non-probe nucleic acid spots. The density of the nucleic acid spots on the solid surface is at least about 5/cm² and usually at least about 10/cm² and may be as high as 1000/cm² or higher, but in many embodiments does not exceed about 1000/cm², and in these embodiments usually does not exceed about 500/cm² or 400/cm², and in certain embodiments does not exceed about 300/cm². The spots may be arranged in a spatially defined and physically addressable manner, in any convenient pattern across or over the surface of the array, such as in rows and columns so as to form a grid, in a circular pattern, and the like, where generally the pattern of spots will be present in the form of a grid across the surface of the solid support.

In the subject arrays, the spots of the pattern are stably associated with or immobilized on the surface of a solid support, where the support may be a flexible or rigid support. By “stably associated” it is meant that the oligonucleotides of the spots maintain their position relative to the solid support under hybridization and washing conditions. As such, the oligonucleotide members which make up the spots can be non-covalently or covalently stably associated with the support surface based on technologies well known to those of skill in the art. Examples of non-covalent association include nonspecific adsorption, binding based on electrostatic (e.g. ion, ion pair interactions), hydrophobic interactions, hydrogen bonding interactions, specific binding through a specific binding pair member covalently attached to the support surface, and the like. Examples of covalent binding include covalent bonds formed between the spot oligonucleotides and a functional group present on the surface of the rigid support, e.g. OH, where the functional group may be naturally occurring or present as a member of an introduced linking group. In many preferred embodiments, the nucleic acids making up the spots on the array surface, or at least the tag complement molecules of the probe spots, are covalently bound to the support surface, e.g. through covalent linkages formed between moieties present on the probes (e.g. thymidine bases) and the substrate surface, etc.

As mentioned above, the array is present on either a flexible or rigid substrate. By flexible is meant that the support is capable of being bent, folded or similarly manipulated without breakage. Examples of solid materials which are flexible solid supports with respect to the present invention include membranes, flexible plastic films, and the like. By rigid is meant that the support is solid and does not readily bend, i.e. the support is not flexible. As such, the rigid substrates of the subject arrays are sufficient to provide physical support and structure to the polymeric targets present thereon under the assay conditions in which the array is employed, particularly under high throughput handling conditions. Furthermore, when the rigid supports of the subject invention are bent, they are prone to breakage.

The solid supports upon which the subject patterns of spots are presented in the subject arrays may take a variety of configurations ranging from simple to complex, depending on the intended use of the array. Thus, the substrate could have an overall slide or plate configuration, such as a rectangular or disc configuration. In many embodiments, the substrate will have a rectangular cross-sectional shape, having a length of from about 10 mm to 200 mm, usually from about 40 to 150 mm and more usually from about 75 to 125 mm and a width of from about 10 mm to 200 mm, usually from about 20 mm to 120 mm and more usually from about 25 to 80 mm, and a thickness of from about 0.01 mm to 5.0 mm, usually from about 0.01 mm to 2 mm and more usually from about 0.01 to 1 mm. Thus, in one representative embodiment the support may have a micro-titre plate format, having dimensions of approximately 125×85 mm. In another representative embodiment, the support may be a standard microscope slide with dimensions of from about 25×75 mm.

The substrates of the subject arrays may be fabricated from a variety of materials. The materials from which the substrate is fabricated should ideally exhibit a low level of non-specific binding during hybridization events. In many situations, it will also be preferable to employ a material that is transparent to visible and/or UV light. For flexible substrates, materials of interest include: nylon, both modified and unmodified, nitrocellulose, polypropylene, and the like, where a nylon membrane, as well as derivatives thereof, is of particular interest in this embodiment. For rigid substrates, specific materials of interest include: glass; plastics, e.g. polytetrafluoroethylene, polypropylene, polystyrene, polycarbonate, and blends thereof, and the like; metals, e.g. gold, platinum, and the like; etc. Also of interest are composite materials, such as glass or plastic coated with a membrane, e.g. nylon or nitrocellulose, etc.

The substrates of the subject arrays comprise at least one surface on which the pattern of spots is present, where the surface may be smooth or substantially planar, or have irregularities, such as depressions or elevations. The surface on which the pattern of spots is present may be modified with one or more different layers of compounds that serve to modify the properties of the surface in a desirable manner. Such modification layers, when present, will generally range in thickness from a monomolecular thickness to about 1 mm, usually from a monomolecular thickness to about 0.1 mm and more usually from a monomolecular thickness to about 0.001 mm. Modification layers of interest include: inorganic and organic layers such as metals, metal oxides, polymers, small organic molecules and the like. Polymeric layers of interest include layers of: peptides, proteins, polynucleic acids or mimetics thereof, e.g. peptide nucleic acids and the like; polysaccharides, phospholipids, polyurethanes, polyesters, polycarbonates, polyureas, polyamides, polyethyleneamines, polyarylene sulfides, polysiloxanes, polyimides, polyacetates, polyacrylamides, and the like, where the polymers may be hetero- or homopolymeric, and may or may not have separate functional moieties attached thereto, e.g. conjugated.

The total number of spots on the substrate will vary depending on the number of different oligonucleotide probe spots (oligonucleotide probe compositions) one wishes to display on the surface, as well as the number of non probe spots, e.g control spots, orientation spots, calibrating spots and the like, as may be desired depending on the particular application in which the subject arrays are to be employed. Generally, the pattern present on the surface of the array will comprise at least about 10 distinct nucleic acid spots, usually at least about 20 nucleic acid spots, and more usually at least about 50 nucleic acid spots, where the number of nucleic acid spots may be as high as 10,000 or higher, but will usually not exceed about 5,000 nucleic acid spots, and more usually will not exceed about 3,000 nucleic acid spots and in many instances will not exceed about 2,000 nucleic acid spots. In certain embodiments, it is preferable to have each distinct probe spot or probe composition be presented in duplicate, i.e. so that there are two duplicate probe spots displayed on the array for a given target. In certain embodiments, each target represented on the array surface is only represented by a single type of oligonucleotide probe. In other words, all of the oligonucleotide probes on the array for a give target represented thereon have the same sequence. In certain embodiments, the number of spots will range from about 200 to 1200. The number of tag complement probe spots present in the array will typically make up a substantial proportion of the total number of nucleic acid spots on the array, where in many embodiments the number of probe spots is at least about 50 number %, usually at least about 80 number % and more usually at least about 90 number % of the total number of nucleic acid spots on the array. As such, in many embodiments the total number of tag complement probe spots on the array ranges from about 50 to 20,000, usually from about 100 to 10,000 and more usually from about 200 to 5,000.

In the arrays of the subject invention (particularly those designed for use in high throughput applications, such as high throughput analysis applications), a single pattern of tag complement spots may be present on the array or the array may comprise a plurality of different tag complement spot patterns, each pattern being as defined above. When a plurality of different tag complement spot patterns are present, the patterns may be identical to each other, such that the array comprises two or more identical tag complement spot patterns on its surface, or the oligonucleotide spot patterns may be different, e.g. in arrays that have two or more different sets of tag complements probes present on their surface, e.g an array that has a pattern of tag complement spots corresponding to first population of tags and a second pattern of tag complement spots corresponding to a second population of tags. Where a plurality of tag complement spot patterns are present on the array, the number of different tag complement spot patterns is at least 2, usually at least 6, more usually at least 24 or 96, where the number of different patterns will generally not exceed about 384.

Where the array comprises a plurality of tag complement spot patterns on its surface, preferably the array comprises a plurality of reaction chambers, wherein each chamber has a bottom surface having associated therewith an pattern of tag complement spots and at least one wall, usually a plurality of walls surrounding the bottom surface. See e.g. U.S. Pat. No. 5,545,531, the disclosure of which is herein incorporated by reference. Of particular interest in many embodiments are arrays in which the same pattern of spots in reproduced in 24 or 96 different reaction chambers across the surface of the array.

Within any given pattern of spots on the array, there may be a single tag complement spot that corresponds to a given tag or a number of different tag complement spots that correspond to the same tag, where when a plurality of different tag complement spots are present that correspond to the same tag, the tag complement probe compositions of each spot that corresponds to the same tag may be identical or different. In other words, a plurality of different tags are represented in the pattern of tag complement spots, where each tag may correspond to a single tag complement spot or a plurality of spots, where the tag complement probe compositions among the plurality of spots corresponding to the same tag may be the same or different. Where a plurality of spots (of the same or different composition) corresponding to the same tag is present on the array, the number of spots in this plurality will be at least about 2 and may be as high as 10, but will usually not exceed about 5. As mentioned above, however, in many preferred embodiments, any given tag is represented by only a single type of tag complement probe spot, which may be present only once or multiple times on the array surface, e.g. in duplicate, triplicate etc.

The number of different tag complements present on the array, and therefore the number of different tags represented on the array, is at least about 2, usually at least about 10 and more usually at least about 20, where in many embodiments the number of different tags represented on the array is at least about 50 and more usually at least about 100. The number of different tags represented on the array may be as high as 5,000 or higher, but in many embodiments will usually not exceed about 3,000 and more usually will not exceed about 2,500. A tag is considered to be represented on an array if it is able to hybridize to one or more tag complement probe compositions on the array.

Additional Features of the Tag-Tag Complement Pairs

The tags and tag complements of the tagged target nucleic acids and arrays, respectively, employed in any given embodiment of subject methods are, in many embodiments, characterized by the following additional features. In many embodiments of the subject invention, any tag or tag complement that is employed is a member of a collection of tag-tag complement pairs in which the hybridization efficiency of each constituent tag-tag complement pair is substantially the same, i.e. all of the tag-tag complement pairs in the population or collection of tag-tag complement pairs are characterized by having substantially the same hybridization efficiency. As such, the hybridization of a tag to its complementary tag complement in any given tag-tag complement pair of the population or collection is substantially the same as that observed for any other given tag-tag complement pair in the population. By substantially the same is meant that the hybridization efficiency is the same or, if it varies, it does not vary by more than about 10 fold, usually by more than about 5 fold and more usually by more than about 3 fold. Hybridization or binding efficiency refers to the ability of the tag complement to bind to its tag under the hybridization conditions in which the array is used. Put another way, binding efficiency refers to the duplex yield obtainable with a given tag complement and its complementary tag after performing a hybridization experiment. In addition to having substantially the same hybridization or binding efficiency, the tag-tag complement pairs are typically further characterized by exhibiting high binding efficiency. In many embodiments, the tag-tag complement pairs present in the population or collection employed in the subject methods exhibit high hybridization efficiency having a binding efficiency of 0.1%, usually at least 0.5% and more usually at least 2% binding of tagget target molecules present in the hybridization assay with the tag complement probe array of the invention.

In addition to exhibiting substantially the same high hybridization efficiency, the tag-tag complement pairs of the collections employed in the subject methods are further chosen to provide for low levels of cross hybridization, i.e. low levels of non-specific hybridization or binding. In other words, the sequence of the tag complement and its corresponding (e.g. complementary) tag are chosen to provide for low non-specific hybridization or non-specific binding, i.e. unwanted cross-hybridization, under stringent conditions. A given tag is considered to be substantially non-complementary to a given tag complement if the tag has homology to the tag complement of less than 60%, more commonly less than 50% and most commonly less than 40%, as determined using the FASTA program with default settings. In certain embodiments, tag-tag complement pairs having low non-specific hybridization characteristics and finding use in the subject methods are those in which the relative ability of the tag or tag complement ability to hybridize to a non-complementary nucleic acid, i.e., other tag complements or tags for which they are not substantially complementary, is less than 10%, usually less than 5 or 2% and preferably less than 1% of their ability to bind to their complementary nucleic acid, i.e. tag or tag complement. For example, in a side-by-side hybridization assay, tag complements having low non-specific hybridization characteristics are those which generate a positive signal, if any, when contacted with a tag composition that does not include a complementary tag for the tag complement, that is less than about 10%, usually least than about 3 or 2% and more usually less than about 1% of the signal that is generated by the same tag complement when it is contacted with a tag composition that includes a complementary tag.

The sequences of the individual tags and tag complements that make up the population of tag-tag complement pairs employed in the subject methods and having the characteristics described above may be determined using any convenient protocol.

In many embodiments, the protocol that is employed identifies sequences that meet the following parameters or criteria. First, the sequence that is chosen as the tag or tag complement sequence should yield a tag-tag complement pair the members of which, i.e. the tag or tag complement, do not cross-hybridize with, or are not homologous to, the members of any other tag-tag complement pair in the collection or population of pairs that is employed. Second, the sequence that is chosen for a given member of a tag-tag complement pair in the population should be chosen such that that member has a low homology to a nucleotide sequence found in any known gene, e.g. any gene whose sequence has been deposited in an accessible electronic database or is going to be analyzed by the universal array. As such, sequences that are avoided include those found in: highly expressed gene products, structural RNAs, repetitive sequences found in the RNA sample to be tested with the array and sequences found in vectors, etc. A further consideration is to select sequences which provide for minimal or no secondary structure, structure which allows for optimal hybridization but low non-specific binding, equal or similar thermal stabilities, and optimal hybridization characteristics. A final consideration is to select sequences that give rise to tag-tag complement pairs that show similar high binding efficiency and low cross-hybridization, as described above. Finally, the sequences of the members of the tag-tag complement constituent members of the population are chosen such that they exhibit substantially the same hybridization efficiency, where the difference in hybridization efficiency between any two tag-tag complement pairs in the population preferably does not exceed about 10 fold, more preferably does not exceed about 5 fold and most preferably does not exceed about 3 fold.

One representative protocol for identifying the sequence of the tags and tag complements that make up the subject populations of tag-tag complement pairs is as follows. First the general length of the tag and tag complements is identified. Generally, the length of tag and tag complements ranges from about 10 to 50, usually from about 15 to 40 and more usually from about 25 to 35 nt. In a given collection, the tag and tag complements may be the same length or of different length, where when there is variation in lengths, the variation is not substantial, such that any difference in length does not exceed about 20, usually does not exceed about 10 and more usually does not exceed about 7 or even 5 nt.

Once a tag/tag complement length is identified, all possible sequences for that length are then determined. For example, where the length is 25 nt and the tags/tag complements are to be polymers of the four naturally occurring dideoxynucleotides, a total of 425 sequences are possible. Generally, these sequence are conveniently determined using a computational means. This initial population of potential sequence is then subjected to the following initial selection or screening steps. In other words, screening criteria are employed for this initial population to exclude non-optimal sequences, where sequences that are excluded or screened out in this step include: (a) those with strong secondary structure or self-complementarity (for example long hairpins); (b) those with very high (more than 70%) or very low (less than 40%) GC content; (c) those with long stretches (usually more than 4 bases) of identical consecutive bases or long stretches (more than 8 nt) of sequences enriched in some bases, purine or pyrimidine stretches or particular motifs, like GAGAGAGA, GAAGAGAA; and the like. This step results in a reduction in the population of candidate sequences.

In the next step, sequences are selected that have similar melting temperatures or thermodynamic stability which will provide similar performance in hybridization assays with target nucleic acids. Of interest is the identification of probes that can participate in duplexes whose differences in melting temperature does not exceed 15, usually not more than 10 and more usually not more than 5° C., as determined under stringent hybridization conditions.

Next, the sequence of all sequences deposited in GenBank are searched in order to select tag/tag complements sequences that are unique and are not homologous to any entry in GenBank, particularly any entry related to phage, viral, prokaryotic, archaebacteria, eukaryotic or other genes which are going to be analyzed on the universal array. A unique sequence is defined as a sequence which at least does not have significant homology to any other sequence on the array. For example, where one is interested in identifying suitable 30 base long tag complement probes, sequences which do not have homology of more than about 80% to any consecutive 30 base segment of any of the potential target sequences are selected. This step typically results in a reduced population of candidate sequences as compared to the initial population of possible sequences identified for each specific target.

The final step in this representative design process is to select from the remaining sequences those sequences which provide for low levels of non-specific hybridization and similar high efficiency hybridization, as described above. This final selection is accomplished by practicing the following steps:

For each potential sequence, a tag complement is synthesized and covalently attached (in similar amount) to a solid surface, thus generating array of tag complements;

A set of control labeled tags is then synthesized and combined, where each of the control tags in the set is present in substantially the same amount as the other control tags. The number of different labeled tags in the control set is usually less than the number of tag complements in the array. Usually the set of control tags is about 50%, more commonly 80% and most commonly 90% from the number of tag complements in the array.

The set of control tags is then hybridized with the tag complement array and hybridization signals for all tag complements are detected. Intensities of signal for tag complements which have labeled complementary tags in hybridization solution (i.e. in the control tag set) reflect efficiency and differences in hybridization of different tags. For the tag complements which do not have complementary tag sequences in the control set, the intensity of hybridization signals reflects the level of non-specific hybridization.

The above steps are then repeated with another set of control tags in order to obtain comprehensive information concerning hybridization efficiency and level of non-specific hybridization for each tag complement in the array.

Using information obtained from the above steps, tag-tag complement pairs are then selected which satisfy the following criteria:

Differences in hybridization efficiency between all selected tag-tag complement pairs in the array are less than 10-fold, more commonly less than 5-fold and most commonly less than 3-fold.

Any tag-tag complement pairs which show level of cross hybridization (non specific hybridization) more than 10%, more commonly 2% and most commonly more than 1% from level of tag-specific hybridization were rejected for further use for the purpose of invention.

The above protocol identifies a set of tag-tag complement pairs that can be employed in the subject methods from an initial set or collection of possible pairs based on the desired length of the tag/tag complement pairs. For example, where one initially has a total of 425 potential sequences and tag-tag complement pairs to choose from, the above protocol allows one to select about 20,000, commonly about 10,000 and more commonly about 5,000 different tag-tag complement pairs, where the identified and selected pairs exhibit similar very efficient hybridization characteristics and minimal levels of non-specific hybridization. The above protocols also provide a number of additional advantages, including: (a) significantly eliminating the need for using theoretical and non-reliable algorithms for tag selection; (b) significantly improving the quality of expression data generated by universal array; (c) simplify data analysis: and (d) significantly reducing the cost of array production.

Non-Tag Complement Probe Spots

In addition to the tag complement spots comprising the tag complement probe compositions (i.e. tag probe spots), the subject arrays may comprise one or more additional nucleic acid spots which do not correspond to target nucleic acids as defined above, such as target nucleic acids of the type or kind of gene represented on the array in those embodiments in which the array is of a specific type. In other words, the array may comprise one or more non-probe nucleic acid spots that are made of non “unique” oligonucleotides or polynucleotides, i.e common oligonucleotides or polynucleotides. For example, spots comprising genomic DNA may be provided in the array, where such spots may serve as orientation marks. Spots comprising plasmid and bacteriophage genes, genes from the same or another species which are not expressed and do not cross hybridize with the cDNA target, and the like, may be present and serve as negative controls. In addition, spots comprising a plurality of oligonucleotides complimentary to housekeeping genes and other control genes from the same or another species may be present, which spots serve in the normalization of mRNA abundance and standardization of hybridization signal intensity in the sample assayed with the array. Orientation spots may also be included on the array, where such spots serve to simplify image analysis of hybrid patterns. Other types of spots include spots for calibration or quantitative standards, controls for integrity of RNA template (targets), controls for efficiency steps in target preparation (such as efficiency of labeling, purification and hybridization), etc. These latter types of spots are distinguished from the tag complement probe spots, i.e. they are non-probe spots.

Array Preparation

The subject arrays can be prepared using any convenient means. One means of preparing the subject arrays is to first synthesize the nucleic acids for each spot and then deposit the nucleic acids as a spot on the support surface. The nucleic acids may be prepared using any convenient methodology, where chemical synthesis procedures using phorphoramidite or analogous protocols in which individual bases are added sequentially without the use of a polymerase, e.g. such as is found in automated solid phase synthesis protocols, and the like, are of particular interest, where such techniques are well known to those of skill in the art.

Following synthesis of the subject tag complement probe molecules, the probes are stably associated with the surface of the solid support. This portion of the preparation process typically involves deposition the probes, e.g. a solution of the probes, onto the surface of the substrate, where the deposition process may or may not be coupled with a covalent attachment step, depending on how the probes are to be stably attached to the substrate surface, e.g. via electrostatic interactions, covalent bonds, etc. The prepared oligonucleotides may be spotted on the support using any convenient methodology, including manual techniques, e.g. by micro pipette, ink jet, pins, etc., and automated protocols. Of particular interest is the use of an automated spotting device, such as the BioGrid Arrayer (Biorobotics).

Where desired, the tag complement molecules can be covalently bonded to the substrate surface using a number of different protocols. For example, functionally active groups such as amino, etc., can be introduced onto the 5′ or 3′ ends of the oligonucleotides, where the introduced functionalities are then reacted with active surface groups on the substrate to provide the covalent linkage. In certain preferred embodiments, the probes are covalently bonded to the surface of the substrate using the following protocol. In this process, the probes are covalently attached to the substrate surface under denaturing conditions. Typically, a denaturing composition of each probe is prepared and then deposited on the substrate surface. By denaturing composition is meant that the probe molecules present in the composition are not participating in secondary structures, e.g. through self-hybridization or hybridization to other molecules in the composition. The denaturing composition, typically a fluid composition, may be any composition which inhibits the formation of hydrogen bonds between complementary nucleotide bases. Thus, compositions of interest are those that include a denaturing agent, e.g. urea, formamide, sodium thiocyanate, etc., as well as solutions having a high pH, e.g. 12 to 13.5, usually 12.5 to 13, or a low pH, e.g. 1 to 4, usually 1 to 3; and the like. In many preferred embodiments, the composition is a strongly alkaline solution of the long oligonucleotide, where the composition comprises a base, e.g. sodium hydroxide, lithium hydroxide, potassium hydroxide, ammonium hydroxide, tetramethyl ammonium hydroxide, ammonium hydroxide, etc, in sufficient amounts to impart the desired high pH to the composition, e.g. 12.5 to 13.0. In another embodiment, high salt concentrations are employed, e.g., 0.5 to 2M LiCl, 2×SSC, 0.5 to 1M NaHCO₃, etc. Detergents, e.g., 0.01 to 0.1% SDS, etc., may also be employed. The concentration of long oligonucleotide in the composition typically ranges from about 0.1 to 10 μM, usually from about 0.5 to 5 μM. Following deposition of the denaturing composition of the long oligonucleoide probe onto the substrate surface, the deposited probe is exposed to UV radiation of sufficient wavelength, e.g. from 250 to 350 nm, to cross link the deposited probe to the surface of the substrate. The irradiation wavelength for this process typically ranges from about 50 to 1000 mJoules, usually from about 100 to 500 mJoules, where the duration of the exposure typically lasts from about 20 to 600 sec, usually from about 30 to 120 sec. In yet other embodiments, non-denaturing conditions are employed for the deposition portion of the protocol.

The above protocol for covalent attachment results in the random covalent binding of the probe to the substrate surface by one or more attachment sites on the probe, where such attachment may optionally be enhanced through inclusion of oligo dT regions at one or more ends of the probes, as discussed supra. An important feature of the above process is that reactive moieties, e.g. amino, that are not present on naturally occurring probes are not employed in the subject methods. As such, the subject methods are suitable for use with probes that do not include moieties that are not present on naturally occurring nucleic acids.

The above described covalent attachment protocol may be used with a variety of different types of substrates. Thus, the above described protocols can be employed with solid supports, such as glass, plastics, membranes, e.g. nylon, and the like. The surfaces may or may not be modified. For example, the nylon surface may be charge neutral or positively charged, where such substrates are available from a number of commercial sources. For glass surfaces, in many embodiments the glass surface is modified, e.g. to display reactive functionalities, such as amino, phenyl isothiocyanate, etc.

Hybridization Methods

As summarized above, the subject methods are hybridization assays in which the tagged target nucleic acids are contacted with a tag complement array, i.e. a universal array of tag complements. In many embodiments, the tagged target nucleic acids that are hybridized to the array are single stranded nucleic acids, such that the hybridized array is an array of duplex structures of hybridized tag and tag complement domains and single stranded target domains.

In practicing the subject methods, following preparation of the tagged target nucleic acid population (usually labeled) from the initial sample and set of tagged gene specific primers, as described supra, the population of tagged target nucleic acids is then contacted with the tag complement or universal array under hybridization conditions, where such conditions can be adjusted, as desired, to provide for an optimum level of specificity in view of the particular assay being performed. Suitable hybridization conditions are well known to those of skill in the art and reviewed in Maniatis et al, supra and WO 95/21944.

Of particular interest in many embodiments is the use of stringent conditions during hybridization, i.e. conditions that are optimal in terms of rate, yield and stability for specific tag-tag complement hybridization and provide for a minimum of non-specific tag-tag complement interaction. Stringent conditions are known to those of skill in the art. In the present invention, stringent conditions are typically characterized by temperatures ranging from 15 to 35, usually 20 to 30° C. less than the melting temperature of the probe target duplexes, which melting temperature is dependent on a number of parameters, e.g. temperature, buffer compositions, size of probes and targets, concentration of probes and targets, etc. As such, the temperature of hybridization typically ranges from about 20 to 70, usually from about 25 to 60° C. The stringent hybridization conditions are further typically characterized by the presence of a hybridization buffer, where the buffer is characterized by one or more of the following characteristics: (a) having a high salt concentration, e.g. 3 to 6×SSC (or other salts with similar concentrations); (b) the presence of detergents, like SDS (from 0.1 to 20%), triton X100 (from 0.01 to 1%), Nonidet NP40 (from 0.1 to 5%) etc.; (c) other additives, like EDTA (typically from 0.1 to 1 μM), tetramethylammonium chloride; (d) accelerating agents, e.g. PEG, dextran sulfate (5 to 10%), CTAB, SDS and the like; (e) denaturing agents, e.g. formamide, urea (0.5 to 6 M) etc.; and the like.

In analyzing the differences in the population of tagged labeled target nucleic acids generated from two or more physiological sources using the arrays described above, in certain embodiments each population of labeled target nucleic acids are separately contacted to identical probe arrays or together to the same array under conditions of hybridization, preferably under stringent hybridization conditions, such that labeled target nucleic acids hybridize to complementary probes on the substrate surface. In yet other embodiments, labeled target nucleic acids are combined with a distinguishably labeled standard or control target nucleic acids followed by hybridization of the combined populations to the array surface, as described in application Ser. No. 09/298,361; the disclosure of which is herein incorporated by reference. In yet other embodiments, a sandwich format is employed, in which the tagged target nucleic acids are unlabeled and, either prior to or after hybridization to the universal array, are hybridized to a second labeled nucleic acid complementary to the gene specific portion of the tagged target nucleic acid, which produces detectably labeled sandwich structures on the array surface. See e.g., Maldonado-Rodriquez et al., Mol. Biotechnol. (1999) 11:1-12.

Where all of the target sequences comprise the same label, different arrays will be employed for each physiological source (where different could include using the same array at different times). Alternatively, where the labels of the targets are different and distinguishable for each of the different physiological sources being assayed, the opportunity arises to use the same array at the same time for each of the different target populations. Examples of distinguishable labels are well known in the art and include: two or more different emission wavelength fluorescent dyes, like Cy3 and Cy5, two or more isotopes with different energy of emission, like ³²P and ³³P, gold or silver particles with different scattering spectra, labels which generate signals under different treatment conditions, like temperature, pH, treatment by additional chemical agents, etc., or generate signals at different time points after treatment. Using one or more enzymes for signal generation allows for the use of an even greater variety of distinguishable labels, based on different substrate specificity of enzymes (alkaline phosphatase/peroxidase).

Following hybridization, non-hybridized labeled nucleic acid is removed from the support surface, conveniently by washing, generating a pattern of hybridized nucleic acid on the substrate surface. A variety of wash solutions are known to those of skill in the art and may be used.

The resultant hybridization patterns of labeled nucleic acids may be visualized or detected in a variety of ways, with the particular manner of detection being chosen based on the particular label of the target nucleic acid, where representative detection means include scintillation counting, autoradiography, fluorescence measurement, colorimetric measurement, light emission measurement, light scattering, and the like.

Following detection or visualization, the hybridization patterns may be compared to identify differences between the patterns. Where arrays in which each of the different probes corresponds to a known gene are employed, any discrepancies can be related to a differential expression of a particular gene in the physiological sources being compared.

The provision of appropriate controls on the arrays permits a more detailed analysis that controls for variations in hybridization conditions, cross-hybridization, nonspecific binding and the like. Thus, for example, in a preferred embodiment, the hybridization array is provided with normalization controls. These normalization controls are complementary to probe tag sequences present on the array prepared separately and added in a known concentration to the labeled tagged target sample both labeled by different labels. Where the overall hybridization conditions are poor, the normalization controls will show a smaller signal reflecting reduced hybridization. Conversely, where hybridization conditions are good, the normalization controls will provide a higher signal reflecting the improved hybridization. Normalization of the signal derived from other probes in the array to the normalization controls thus provides a control for variations in hybridization conditions. Normalization control is also useful to adjust (e.g. correct) for differences which arise from the array quality, the mRNA sample quality, efficiency of first-strand synthesis, etc. Typically, normalization is accomplished by dividing the measured signal from the other probes in the array by the average signal produced by the normalization controls. Normalization may also include correction for variations due to sample preparation and amplification. Such normalization may be accomplished by dividing the measured signal by the average signal from the sample preparation/amplification control targets. The resulting values may be multiplied by a constant value to scale the results.

In certain embodiments, normalization controls are often unnecessary for useful quantification of a hybridization signal. Thus, where optimal probes have been identified, the average hybridization signal produced by the selected optimal probes provides a good quantified measure of the concentration of hybridized nucleic acid. However, normalization controls may still be employed in such methods for other purposes, e.g. to account for array quality, mRNA sample quality, etc.

Although the above described methods have been presented in terms of contacting the tagged target nucleic acids with the tag complement or universal array, one can also cleave the tag portion from the target nucleic acid portion of the tagged target nucleic acids prior to contact with the array, since the cleaved tags are representative of the target nucleic acids in the tagged target nucleic acid population.

By way of further illustration, the following representative gene expression assay is summarized. Where one is interested in assaying a sample for the presence of 100 different mRNAs, a collection of 100 different tagged gene specific primers is prepared, where each different tagged gene specific primer in the collection hybridizes to a different mRNA member of the 100 different proteins being assayed. The collection of 100 different tagged gene specific primers is used to generate labeled, tagged target nucleic acids for any of the 100 mRNAs of interest that are present in the sample. The resultant tagged target nucleic acids are then hybridized to a universal array of tag complements and the resultant surfaces bound duplexes are detected and the location of the detected surface bound duplexes is used to determine which of the 100 mRNAs of interest is present in the sample, and therefore which the 100 genes corresponding to the 100 mRNAs is expressed in the cell from which the sample was derived. In order to increase specificity, a second detection probe can be employed. See e.g., the sandwich detection protocol described above.

Utility

The subject methods find use in, among other applications, differential gene expression assays. Thus, one may use the subject methods in the differential expression analysis of: (a) diseased and normal tissue, e.g. neoplastic and normal tissue, (b) different tissue or tissue types; (c) developmental stage; (d) response to external or internal stimulus; (e) response to treatment; (f) different strains of microorganisms or viruses; and the like. The subject arrays therefore find use in broad scale expression screening for drug discovery, diagnostics and research, as well as studying the effect of a particular active agent on the expression pattern of genes in a particular cell, where such information can be used to reveal drug toxicity, carcinogenicity, etc., environmental monitoring, infection/disease research and the like.

The subject methods provide for a significant advantage over other array based hybridization assays in the above described and other applications. Specifically, the subject methods are based on the use of a universal array of tag complements, i.e. an array that is not specifically tailored to detection of specific genes in a sample. Instead, specificity with regard to the types of genes that are assayed by the arrays is provided by attaching the tags to the desired gene specific primers and using the tagged gene specific primers in the target generation portion of the assay. As such, one can use the same universal array and corresponding set of tags in any gene expression assay, with the specificity of genes assayed being provided by at least the gene specific primer portions that are employed.

Kits

Also provided are kits for performing hybridization assays according to the subject invention. Such kits according to the subject invention include at least one of: (a) a tag complement or universal array; and (b) a set of tagged gene specific primers, where the tag portion of each member of the set of gene specific primers corresponds to, i.e. is complementary to or has a sequence identical to a sequence found in, a tag complement on the array. In many embodiments, the kits include both the universal array and a set of tagged gene specific primers.

In addition to including at least one of the array and the set of tagged gene specific primers, the kits also include a means for determining the gene to which each tag and tag complement on the array corresponds. In other words, the kits include a means for readily matching any given tag and tag complement pair with a specific gene. Put another way, the kits include a means for readily identifying the location on the array that a specific tagged gene specific primer, and therefore tagged target nucleic acid prepared therefrom, will hybridize during a hybridization assay. With this means, one can readily identify the location on the array that corresponds to a particular gene of interest in the assay that is to be performed.

This means for identifying the gene to which a given tag-tag complement pair correspond may take a variety of forms, one or more of which may be present in the kit. One form in which this means may be present is as printed information on a suitable medium or substrate, e.g. a piece or pieces of paper on which the information is printed. Yet another means would be a computer readable medium, e.g. diskette, CD, etc., on which the information has been recorded. Yet another means that may be present is a website address which may be used via the internet to access the information at a removed site. Any convenient means may be present in the kits.

The kits may further comprise one or more additional reagents employed in the various methods, such as normalization controls, primers for generating target nucleic acids, dNTPs and/or rNTPs, which may be either premixed or separate, one or more uniquely labeled dNTPs and/or rNTPs, such as biotinylated or Cy3 or Cy5 tagged dNTPs, gold or silver particles with different scattering spectra, or other post synthesis labeling reagent, such as chemically active derivatives of fluorescent dyes, enzymes, such as reverse transcriptases, DNA polymerases, RNA polymerases, and the like, various buffer mediums, e.g. hybridization and washing buffers, prefabricated probe arrays, labeled probe purification reagents and components, like spin columns, etc., signal generation and detection reagents, e.g. streptavidin-alkaline phosphatase conjugate, chemifluorescent or chemiluminescent substrate, and the like.

It is evident from the above discussion that the methods provide for a significant advance in the field. The subject invention provides for the use of a single “universal array” in a plurality of different gene expression assays which differ from each other with respect to the identity of the genes being assayed. The same universal array can be manufactured and used in many different types of hybridization assays, thereby providing for ease in quality control, high throughput manufacture, and economical manufacture. Accordingly, the subject invention represents a significant contribution to the art.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. A hybridization assay comprising the steps of:
(a) generating a population of tagged target nucleic acids from an initial sample of nucleic acids with a collection of a representative number of tagged gene specific primers;
(b) contacting said population of tagged target nucleic acids with an array of tag complements immobilized on a solid support; and
(c) detecting any resultant hybridization complexes on said array.

2. The hybridization assay according to claim 1, wherein said tagged gene specific primers are not used in an amplification step.

3. The hybridization assay according to claim 1, wherein the magnitude of any difference in hybridization efficiency between any two tag-tag complement pairs employed in said assay does not exceed about 10 fold.

4. The hybridization assay according to claim 1, wherein any tag employed in said assay has a level of cross-hybridization that does not exceed about 10%.

5. The hybridization assay according to claim 1, wherein said tagged target nucleic acids are labeled.

6. The hybridization assay according to claim 1, wherein said generating step (a) comprises enzymatically generating said population of labeled, tagged target nucleic by a protocol that includes a non-amplification primer extension step in which said collection of a representative number of tagged gene specific primers is employed.

7. The hybridization assay according to claim 6, wherein the magnitude of any difference in hybridization efficiency between any two tag-tag complement pairs employed in said assay does not exceed about 5 fold.

8. The hybridization assay according to claim 7, wherein the magnitude of any difference in hybridization efficiency between any two tag-tag complement pairs employed in said assay does not exceed about 3 fold.

9. The hybridization assay according to claim 8, wherein any tag employed in said assay has a level of cross-hybridization that does not exceed about 2%.

10. The hybridization assay according to claim 9, wherein any tag employed in said assay has a level of cross-hybridization that does not exceed about 1%.

11. The hybridization assay according to claim 6, wherein said initial nucleic acid sample is a ribonucleic acid sample.

12. The hybridization assay according to claim 6, wherein said assay comprises generating labeled, tagged target nucleic acids from at least two distinct initial nucleic acid samples.

13. A kit for use in a hybridization assay, said kit comprising:
(a) at least one of:
(i) an array of distinct tag complements immobilized on the surface of a solid support; and
(ii) a set of a representative number of distinct tagged gene specific primers; and
(b) means for identifying the physical location on said array to which each distinct tagged gene specific primer hybridizes.

14. The kit according to claim 13, wherein said kit comprises both said array and said set of tagged gene specific primers.

15. The kit according to claim 13, wherein the magnitude of any difference in hybridization efficiency between any two tag-tag complement pairs taken from said array and set of tagged gene specific primers does not exceed about 10 fold.

16. The kit according to claim 13, wherein any tag found in said set of tagged gene specific primers has a level of cross-hybridization with respect to said array that does not exceed about 10%.

17. The kit according to claim 13, wherein said means comprises a medium that includes: (a) identifying information about the physical location on said array to which each distinct tagged gene specific primer hybridizes; or (b) a means for remotely accessing said information.

18. The kit according to claim 17, wherein said means for remotely accessing said information is a website address.

19. An array of distinct tag complements immobilized on a solid support, wherein said tag complements are members of a collection of tag-tag complement pairs in which the magnitude of any difference in hybridization efficiency between any two tag-tag complement pairs in said collection does not exceed about 10 fold.

20. The array according to claim 19, wherein said tag complements are nucleic acids.

21. The array according to claim 19, wherein said array has a density that does not exceed about 400 spots/cm².

22. A set of a representative number of distinct tagged gene specific primers comprising a tag domain and a primer domain, wherein said tag domains are members of a collection of tag-tag complement pairs in which the magnitude of any difference in hybridization efficiency between any two tag-tag complement pairs in said collection does not exceed about 10 fold.

23. The set according to claim 22, wherein each gene specific primer is a deoxyribonucleic acid.

24. The set according to claim 22, wherein any tag domain has a level of cross-hybridization with respect to said tag complements of said collection that does not exceed about 10%.

25. The set according to claim 22, wherein said set comprises at least 20 distinct tagged gene specific primers.
